(19) United States
(12) Patent Application Publication
(10) Pub. No.: US 2006/0270843 A1
(43) Pub. Date: Nov. 30, 2006

(54) Methods for isolation of nucleic acids

(76) Inventors: Gerald E. Hall JR., Morrisville, PA (US); Barry E. Boyes, Wilimington, DE (US)

Correspondence Address: AGILENT TECHNOLOGIES, INC.;Legal Department, DL 429, Intellectual Property Administration, P.O. Box 7599, Loveland, CO, 80537-0599, US

(21) Appl. No.: 11/140,389

(22) Filed: May 26, 2005

(51) Int. Cl. C07H 21/04

(52) U.S. Cl. 536/25.4

(57) ABSTRACT

This invention relates to methods and kits for collecting different biopolymers from a single sample, such as RNA and genomic DNA. The methods and kits can be used for generating targets for array-based assays such as gene expression assays and comparative genome hybridization assays which can be performed in parallel on the same or different arrays.

### BACKGROUND

High throughput techniques such as microarray-based assays make it possible to analyze a plurality of biopolymer samples in parallel. It is often desirable to characterize different types of biopolymers (e.g., such as DNA, RNA, proteins, etc.) from a single sample to evaluate coordinate changes in such biopolymers and correlate these with a physiological state of an organism or a biological response. While techniques exist for the isolation of RNA or DNA very few options are available for the isolation of both RNA and DNA from the same sample. The most commonly used procedure for the simultaneous isolation of RNA and DNA is described in U.S. Pat. No. 5,346,994. The method requires the use of an extraction solution comprising a chaotropic agent and phenol. Upon separation of the organic and aqueous phases by centrifugation, the RNA partitions with the aqueous phase while DNA remains at the interface. The aqueous phase and interface are isolated and the nucleic acids are further purified. The disadvantages of the method include the use of noxious, toxic chemicals (e.g., phenol and/or chloroform), tedious manipulations (e.g., separation of the aqueous and organic phases and the DNA-containing interfaces), the likelihood of cross-contamination as typically RNA purified using this method is heavily contaminated with DNA and DNA is heavily contaminated with RNA.

The availability of non-organic based isolation methods for the simultaneous isolation of DNA and RNA from a biological source is limited. Examples include the Qiagen RNA/DNA mini Kit, catalog number 14121, and BD Biosciences Nucleobond RNA/DNA Kit, catalog 635945, both of which rely on the use which on the use of anion exchange technology requiring low salt binding and high salt elution. The use of high salt in the elution buffer can interfere with subsequent procedures in which the nucleic acids are used.

### SUMMARY

The invention relates to methods for isolating a plurality of biopolymers from a sample.

In one embodiment, the invention provides a method for separating RNA and DNA (e.g., such as genomic DNA) from a sample. In one aspect, the method comprises contacting a nucleic acid separation material with a sample comprising RNA and DNA, under conditions where DNA is captured by the nucleic acid separation material and RNA is not, wherein the separation conditions include a pH of less than 8.0; removing the nucleic acid separation material from the sample; releasing DNA from the nucleic acid separation material and purifying released DNA.

In certain aspects, the method further comprises purifying RNA remaining in the sample.

In certain aspects, the separation conditions include contacting the sample to the nucleic acid separation material in the absence of alcohol.

In one aspect, the nucleic acid separation material comprises one or more of a silica-based solid phase material.

In certain aspects, DNA is released from the nucleic acid separation material by contacting the nucleic acid separation material with a detergent, for example, an ionic detergent such as N-lauroylsarcosine. In certain aspects, the concentration of N-lauroylsarcosine is about 0.01% to about 5% v/v.

The released DNA may be subjected to one or more purification procedures. Similarly, the RNA remaining in the sample may be subjected to one or more purification procedures. Any purification procedure known in the art can be performed. In one aspect, purification is by precipitation or by contact with a nucleic acid capture material. The nucleic acid capture material for purification may be a silica-based material or a non-silica-based material. In one aspect, the nucleic acid capture material is a polymeric material. In certain aspects, the polymeric material comprises polysulfone. In certain aspects, the polymeric material further comprises polyvinylpyrrolidone.

After contacting with the nucleic acid capture material for purification, the method may further comprise the step of releasing the nucleic acid from the nucleic acid capture material.

In certain aspects, the sample comprises a biological sample and is homogenized prior to contacting the nucleic acid separation material. For example, the sample can be homogenized in a solution comprising a chaotropic salt, e.g., a solution of at least about 4M chaotropic salt, such as, but not limited to, guanidine isothiocyanate, guanidine HCl, sodium perchlorate, ammonium thiocyanate, sodium iodide, or a combination thereof.

In another embodiment, the invention relates to kits for facilitating any of the above methods. For example, in one aspect, the invention relates to a kit comprising a silica-based nucleic acid separation material and an ionic detergent, such as N-lauroylsarcosine. In certain aspects, the kit further comprises a DNA capture material for purifying DNA. In certain aspects, the kit further comprises an RNA capture material for purifying RNA. The DNA and/or RNA capture material can comprise a silica-based material or a non-silica-based material (e.g., such as a polymeric membrane). The DNA and RNA capture materials may be the same or different. In certain aspects, the capture material for purifying a nucleic acid comprises polysulfone. In another aspect, the capture material further comprises polyvinylpyrrolidone. In still another aspect, the kit may further comprise a chaotropic salt, including, but not limited to, guanidine isothiocyanate, guanidine HCl, sodium perchlorate, ammonium thiocyanate, sodium iodide, or a combination thereof.

### BRIEF DESCRIPTION OF THE FIGURES

The objects and features of the invention can be better understood with reference to the following detailed description and accompanying drawings. The Figures shown herein are not necessarily drawn to scale, with some components and features being exaggerated for clarity.

FIG. 1 is a flow diagram illustrating a method according to one aspect of the invention for isolation of RNA and DNA from the same sample.

FIG. 2 is a graph showing recovery of tobacco leaf gDNA from 4-layer glass fiber prefilters using different elution solutions.

FIG. 3 is a graph showing recovery of RNA and DNA from mouse liver extracts using methods according to aspects of the invention.

FIG. 4 shows results of scans of RNA and DNA isolates obtained using methods according to the invention. The left panel shows analysis using an Agilent Bioanalyzer 2100 DNA chip. The right panel shows analysis using an RNA 6000 Nano Chip.

FIG. 5 is a graph showing recovery of gDNA from various mouse tissues using 2-, 4-, and 8-layer glass fiber prefilters according to one aspect of the invention.

FIG. 6 is a photograph showing gel electrophoresis analysis of recovered gDNA. DNA samples were run on a 0.8% agarose gel in 1×TBE.

### DETAILED DESCRIPTION

The present invention pertains to methods and reagents used for collecting and/or isolating subcellular components. In one embodiment, the device is used to separate RNA and DNA in a sample and collect (e.g., purify) RNA and DNA from the same sample. In one aspect, a plurality of RNA isolation steps and DNA isolation steps are performed in parallel. The methods can be used for preparing targets for analysis of gene expression and genome-wide analysis of regulatory events (e.g., binding of DNA binding factors) and copy number. In one embodiment, the methods are used to prepare target nucleic acids for binding to arrays for performing gene expression analysis and a genome assay, e.g., such as comparative genomic hybridization, and the like.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

It must be noted that as used herein and in the appended claims, the singular forms “a”, “an”, and “the” include plural referents unless the context clearly dictates otherwise.

“May” refers to optionally.

When two or more items (for example, elements or processes) are referenced by an alternative “or”, this indicates that either could be present separately or any combination of them could be present together except where the presence of one necessarily excludes the other or others.

The following definitions are provided for specific terms, which are used in the following written description.

The term “binding” refers to two molecules associating with each other to produce a stable composite structure under the conditions being evaluated (e.g., such as conditions suitable for RNA or DNA isolation). Such a stable composite structure may be referred to as a “binding complex”.

As used herein, the term “RNA” or “oligoribonucleotides” refers to a molecule having one or more ribonucleotides. The RNA can be single, double or multiple-stranded (e.g., comprise both single-stranded and double-stranded portions) and may comprise modified or unmodified nucleotides or non-nucleotides or various mixtures and combinations thereof.

As used herein, the term “DNA” or “deoxyribonucleotides” refers to a molecule comprising one or more deoxyribonucleotides. The DNA can be single, double or multiple-stranded (e.g., comprise both single-stranded and double-stranded portions) and may comprise modified or unmodified nucleotides or non-nucleotides or various mixtures and combinations thereof.

As used herein “complementary sequence” refers to a nucleic acid sequence that can form hydrogen bond(s) with another nucleic acid sequence by either traditional Watson-Crick or other non-traditional types (for example, Hoogsteen type) of base-paired interactions.

In certain embodiments, two complementary nucleic acids may be referred to as “specifically hybridizing” to one another. The terms “specifically hybridizing,” “hybridizing specifically to” and “specific hybridization” and “selectively hybridize to,” are used interchangeably and refer to the binding, duplexing, complexing or hybridizing of a nucleic acid molecule preferentially to a particular nucleotide sequence under stringent conditions. “Hybridizing” and “binding”, with respect to polynucleotides, are used interchangeably.

The term “reference” is used to refer to a known value or set of known values against which an observed value may be compared.

It will also be appreciated that throughout the present application, that words such as “cover”, “base” “front”, “back”, “top”, “upper”, and “lower” are used in a relative sense only.

As used herein, the term “solid phase” or “solid substrate” or “matrix” includes rigid and flexible solids. Examples of solid substrates include, but are not limited to, gels, fibers, whiskers, resins, microspheres, spheres, cubes, particles of other shapes, channels, microchannels, capillaries, walls of containers, membranes and filters.

As used herein, the term “silica-based” is used to describe SiO₂ compounds and related hydrated oxides and does not encompass silicon carbide compositions, which are described herein.

As used herein, a “nucleic acid binding material”, stably binds a nucleic acid (e.g., such as double-stranded, single-stranded or partially double-stranded DNA, RNA or modified form thereof). By “stably binds” it is meant that under defined binding conditions the equilibrium substantially favors binding over release of the subcellular component, and if the solid substrate containing a selected bound subcellular component is washed with buffer lacking the component under these defined binding conditions, substantially all the component remains bound. In particular embodiments the binding is reversible. As used herein, the term “reversible” means that under defined elution conditions the bound nucleic acid component of a sample is predominantly released from the nucleic acid binding material and can be recovered (e.g., in solution). In particular embodiments, at least about 10%, at least about 20%, at least about 50%, at least about 60%, at least 90%, or at least 95% of the bound nucleic acid component is released under the defined elution conditions.

As used herein, a “nucleic acid capture material” is one which preferentially retains, or traps, or remains associated with nucleic acids to remove a nucleic acid from solution. A nucleic acid capture material may, but does not necessarily, bind to a nucleic acid molecule.

“Washing conditions” include conditions under which unbound or undesired components are removed from a module of a device described below.

The term “assessing” “inspecting” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

A chemical “array”, unless a contrary intention appears, includes any one, two or three-dimensional arrangement of addressable regions bearing a particular chemical moiety or moieties (for example, biopolymers such as polynucleotide sequences) associated with that region. For example, each region may extend into a third dimension in the case where the substrate is porous while not having any substantial third dimension measurement (thickness) in the case where the substrate is non-porous. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). Such a region may be referred to as a “feature region”. The target for which each feature is specific is, in representative embodiments, known. An array feature is generally homogenous in composition and concentration and the features may be separated by intervening spaces (although arrays without such separation can be fabricated).

Additional terms relating to arrays and the hybridization of nucleic acids to such arrays may be found, for example, in U.S. Pat. No. 6,399,394.

In one embodiment, the invention relates to the use of a device that comprises a module for separating DNA components of a sample from RNA components (“separation module”). In certain aspects, the DNA components comprise genomic DNA, which may or may not have been previously crosslinked (e.g., to DNA binding proteins), fragmented, amplified, and/or labeled. In certain aspects, DNA is reverse transcribed from RNA in the sample. Similarly, RNA can be isolated directly from a sample or after one or more further processing steps, such from in vitro transcription of DNA templates in the sample, amplification, and/or labeling steps. As used herein, the term “module” refers to a functional element or unit in the device that may or may not be removable from the device. The separation module preferentially retains DNA under nucleic acid separation conditions while allowing RNA-containing sample to flow through.

In one aspect, nucleic acid separation conditions include an absence of alcohol. In another aspect, nucleic acid separation conditions includes contacting the nucleic acid separation material with a sample solution having a pH of less than 8.0, e.g., such as from about pH 6.5 to about pH 7.5.

In one embodiment, the separation module comprises a nucleic acid separation material for preferentially retaining DNA while RNA-containing sample flows through under these conditions. The nucleic acid separation material can comprise one or more filters or layers of beads or other type of matrix. For example, in one aspect, the nucleic acid separation material comprises a fibrous, whisker, porous, or polymeric material or combination thereof. Suitable materials include, but are not limited to, glass fibers or borosilicate fibers, silica-based materials, polymers (e.g., beads, filters, membranes, fibers) and the like. In certain aspects, the nucleic acid separation material retains DNA under DNA capture conditions, which may include binding at pHs lower than pH 8.0 and in certain aspects, in the absence of alcohol and/or in the presence of a chaotropic agent.

In one aspect, the nucleic acid separation material comprises a fiber material that demonstrates particle retention in the range of about 0.1 μm to about 10 μm diameter equivalent. The fibers can have a thickness ranging from about 50 μm to about 2,000 μm. For example, in one aspect, a fiber filter has a thickness of about 500 μm. The specific weight of a fiber filter can range from about 75 g/m² up to about 300 g/m². Multiple fiber layers are envisaged to be within the scope of this invention. The fiber may, optionally, comprise a binder, e.g., for improving handling of the fiber or for modifying characteristics of a composite fiber (i.e., one which is not pure borosilicate). Examples of binders include, but are not limited to, polymers such as acrylic, acrylic-like, or plastic-like substances. Although it can vary, typically binders may represent about 5% by weight of the fiber filter.

The pore size of the filter may be uniform or non-uniform. Where a plurality of filters are used, the pore size of each filter may be the same or different. In another aspect, suitable pore sizes may range from about 0.1 μm to about 2 mm.

In a particular aspect of this invention, the separation module comprises a nucleic acid separation material comprising at least one layer of fiber filter material. The filter material can be disposed adjacent to a retainer ring that is adjacent to a first surface of the fiber filter material so that the filter material does not excessively swell when sample is added. In one aspect, a frit is provided which is disposed adjacent to a second surface of the fiber filter material. The frit may assist in providing support so that the materials of the filter fibers do not deform. In one aspect, the frit is composed of polyethylene of about 90 μm thick. In certain aspects, the separation module comprises at least two layers of filter material, at least three layers, at least four layers, at least five layers, at least six layers, at least seven layers, at least 8 layers, at least 9 layers, or at least 10 layers.

In one embodiment, the nucleic acid separation material in the separation module comprises glass fiber filters or an equivalent material. Multiple layers (of the large sheets or disks supplied) may be punched, for example, and placed into a spin column fitted with a polyethylene frit on which the fibers may rest. The filter materials may be secured in the column with a retainer ring on top of the filter materials to prevent excessive swelling of the fibers or movement during centrifugation. In one aspect, the separation module that is used is the prefiltration column available in Agilent's Total RNA Isolation Mini Kit prefiltration column (Catalog #5185-6000) from Agilent Technologies, Inc. (Palo Alto, Calif.).

In one aspect, the separation module does not comprise a matrix for anion exchange.

The configuration of the device comprising the separation module can vary. In one aspect, the device comprises a housing having an open end and comprises walls defining a lumen into which the separation module fits. In another aspect, the device comprises a closed bottom end. The separation module may be removable from the housing or an integral part of the housing or some combination thereof. The shape and dimensions of the housing may vary. However, in one embodiment, the housing is shaped like a tube or column. In another aspect, the housing is shaped like a tube and the separation module is provided in the form of a column that fits into the tube, the remaining space defining a collection compartment or chamber for receiving flow through or molecules eluted from the separation module.

In certain aspects, a plurality of device housings is provided in a holder or container or rack and a plurality of separation modules (e.g., columns) may be inserted into the lumen of each of the housings. In one aspect, the plurality of device housings is provided as a single unit (e.g., molded as a single unit from a plastic or other suitable material) comprising a plurality of lumens for receiving a plurality of columns.

Individual separation modules may be separated from each other one at a time, e.g., by unscrewing or snapping apart. Likewise, the housing may be made from a variety of materials, including but not limiting to, a polymeric material such as plastic, polycarbonate, polyethylene, PTFE, polypropylene, polystyrene and the like.

RNA which flows through the separation module can be collected within the lumen of the housing between the separation module and the closed end of the same or a different device (i.e., the separation module can be transferred to the housing of a different device). This portion of the device forms the “collection module.” Biopolymers collected in the collection module can be removed from the collection module for further processing steps (e.g., such as purification steps). Additionally, or alternatively, processing steps may occur directly in the same collection module. For example, RNA may be precipitated in the collection module using an appropriate alcohol and salt. More particularly, RNA can be pelleted in the collection module after precipitation using an RNA precipitating material (e.g., such as alcohol, LiCl or another salt, or a solution of guanidine and ethanol). Suitable RNA precipitating materials are known in the art. This precipitate can be collected via, for example, centrifugation. However, in certain aspects, as discussed further below, the collection module comprises an RNA capture module comprising a porous, semiporous or fibrous material for trapping and/or binding RNA, e.g., such as a precipitated form of RNA, which can later be released from the RNA capture module.

In one aspect, the separation module is provided in the form of a column that fits into the lumen defined by the walls of the device housing and the collection module is formed in the space between the column and the closed bottom end of the housing. Removing the column from the device provides access to the collection module. Alternatively, the collection module may be removed from the device (e.g., by snapping off or twisting). In one aspect, the closed bottom end may comprise a cap or cover which may be removed to obtain collected material. RNA may be obtained from the collection module for further processing (e.g., such as alcohol precipitation or purification by some other method).

In still other embodiments, RNA-containing flow through from a separation module is collected in a collection module and contacted with an RNA capture material (e.g., in the form of a membrane, matrix, gel, particles, beads, filter, column, and the like) in the same or in a different collection module, for specifically capturing RNA, e.g., to further purify RNA from non-RNA components in the flow through. In one aspect, as discussed further below, the RNA capture material comprises a porous, semiporous, or fibrous material (e.g., such as a porous or semiporous polymer membrane, a group of fibers or filters, etc.), which preferentially retains, or traps, or remains stably associated with, RNA under RNA capture conditions. In one aspect, the RNA capture conditions include conditions for precipitating RNA.

In one aspect, RNA-capture material comprises a silicon carbide matrix, e.g., such as silicon carbide fibers or whiskers. In another aspect, the RNA capture material comprises silica carbide whiskers which comprise a comparatively high specific surface area material, greater than about 0.4 m²/g, greater than 1 m²/g, greater than 2 m²/g, greater than 3 m²/g or about 3.9 m²/g as measured by surface Nitrogen absorption.

In certain aspects, the RNA capture material does not comprise silica.

In still another aspect, the RNA capture material comprises one or more polymeric membranes, examples of which include, but are not limited to, polysulfone, e.g., such as a BTS membrane (Pall Life Sciences), PVDF, nylon, nitrocellulose, and composites thereof. In one aspect, the membrane is a composite of Polysulfone and PVP, such as an MMM filter (Pall Life Sciences, available from VWR, Pittsburg Pa.). In another aspect, the binding material comprises an asymmetric membrane with pores that gradually decrease in size from the upstream side to the downstream side. In one aspect, the membrane comprises pore sizes from about 0.1 μm to 100 μm. In another aspect, the membrane comprises pore sizes of from about 0.1 μm to 10 μm, or from about 0.1 μm to about 1 μm, or from about 0.4 μm to about 0.8 μm. For example, in one aspect, the first surface has 30-40 μm diameter pores and the second surface has 0.1-5.0 μm diameter pores, or 0.4-0.8 μm diameter pores. In another aspect, the membrane comprises intermediate sized pores between the first and second surface. In still another aspect, the larger diameter pores are on the upper side of the membrane while the smaller diameter pores (proximal to the collection module of the device) are on the lower surface.

In still another aspect, the binding material comprises a hydrophobic and/or hydrophilic material.

It should be noted that in certain aspects or under certain conditions, the RNA capture material, does not necessarily bind the nucleic acid (e.g., RNA) but can serve as a physical barrier or trap which prevents precipitated RNA molecules from passing through until they are resuspended or changed from a precipitated to a non-precipitated state, or until the RNA capture material is otherwise contacted with a releasing buffer (e.g., such as a low ionic strength buffer) to change RNA molecules in the sample to a state in which the capture material no longer acts as a physical barrier. In still other aspects, the RNA capture material functions both as a physical barrier and a material to which RNA molecules may bind under the appropriate RNA capture conditions.

In one embodiment, the invention further provides methods of using the devices discussed above to simultaneously obtain different biopolymers from a single sample. In one aspect, the different biopolymers comprise DNA and RNA, which may be obtained directly from a sample or after one or more processing steps as discussed above.

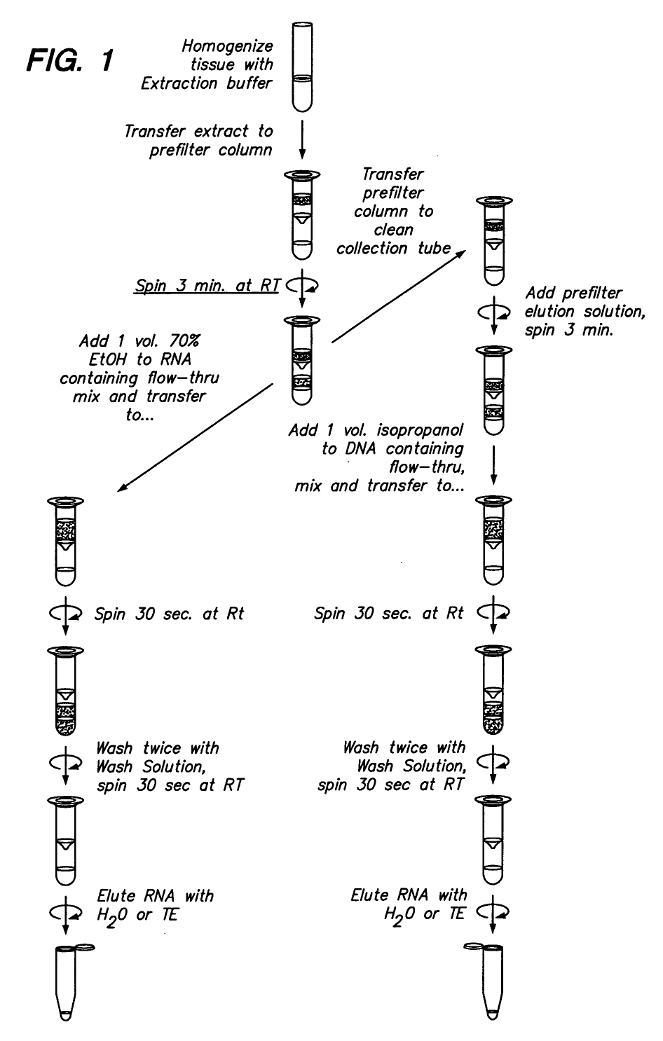

In one embodiment, as illustrated in the Flow Diagram of FIG. 1, a sample is homogenized in an extraction buffer prior to contacting the sample with a separation module comprising a nucleic acid separation material. Sample sources include, but are not limited to animals, plants, fungi (e.g., such as yeast), bacteria, and portions thereof. In one aspect, the animal can be a mammal, and in a further aspect, the mammal can be a human. Sample sources may additionally include virally infected cells, as well as transgenic animals and plants or otherwise genetically modified animals and plants.

Mechanical homogenization can be performed using methods known in the art, e.g., such as by using a rotor-stator homogenizer, such as by grinding in a mortar and pestle with liquid nitrogen, mechanical disruption with a tissue homogenizer, such as a Polytron® or Omniprobe® homogenizer, manual homogenization (e.g., with a Dounce homogenizer), vortexing, and shaking the sample in a container with metal balls. Additionally, or alternatively, samples can be homogenized by ultrasonic disruption. In one aspect, homogenization is done in a high chaotrope concentration solution effectively lysing cells and destroying cellular enzymatic activity, such as the activity of nucleases.

In certain embodiments, samples are lysed before, during, or after homogenization. Suitable lysis solutions are known in the art. However, in one aspect, the lysis solution comprises a chaotropic salt, and/or additives to protect nucleic acids in the sample from degradation or reduced yield. Suitable salts include but are not limited to urea, formaldehyde, ammonium isothiocyanate, guanidinium isothiocyanate, guanidinium hydrochloride, sodium perchlorate, formamide, dimethylsulfoxide, ethylene glycol, tetrafluoroacetate, diamineimine, ketoaminimine, hydroxyamineimine, aminoguanidine hydrochloride, aminoguanidine hemisulfate, hydroxylaminoguanidine hydrochloride, sodium iodide and mixtures thereof. Other additives to protect nucleic acids in the sample from degradation or reduced yield include, but are not limited to, ribonuclease inhibitor, chelating agent, DEPC, vanadyl compound, and mixtures thereof. Examples of ribonuclease inhibitors can be found in Farrell R. E. (ed.) (RNA Methodologies: A Laboratory Guide for Isolation and Characterization, Academic Press, 1993) and Jones, P. et al. (In: RNA Isolation and Analysis, Bios Scientific Publishers, Oxford, 1994). In one aspect, RNAlater® (Ambion Inc., Austin, Tex., U.S. Pat. No. 6,204,375) is used as an RNase inhibitor. In one aspect, an RNase inhibitor inhibits one or more of RNase A, B, C, RNase T1 and RNase 1.

In another aspect, the lysis solution comprises one or more enzymes to facilitate disruption of cells in a sample. Suitable enzymes include, but are not limited to, a protease, lysozyme, zymolase, cellulase, and the like. However, in certain aspects, the lysis solution/extraction solution does not comprise a detergent.

In one embodiment, a lysis solution comprising from about 4 M guanidine salt to about 6 M guanidine salt is employed. In one particular embodiment, the solution comprises 4M guanidine isothiocyanate, 25 mM Tris pH 7.5, 10 mM EDTA, 1% β-mercaptoethanol). In another particular embodiment, the solution comprises 5.5 M guanidine HCl, 50 mM Bis-Tris pH 6.6, 10 mM EDTA, 1% β-mercaptoethanol.

Flow-through obtained after centrifugation or application of pressure or vacuum to the device, or alternatively, through the use of gravity, is collected within the collection module of the device. RNA can be obtained from the flow through in the collection module by precipitation, e.g., by adding alcohol to the flow-through mix to a final concentration of 30-75% and collecting the RNA by centrifugation or by further contacting with an RNA capture material in the same or in a different collection module. In one aspect, as discussed above, alcohol is not added to the sample until it has contacted and flowed through the separation module, i.e., nucleic acid separation conditions do not include the use of alcohol. In certain aspects, RNA capture on the RNA capture material occurs in greater than 1M concentration of a chaotropic salt, greater than 3 M concentration, or at a concentration of about 4M to 6M such as provided in the initial lysis/extraction solution. In certain aspects, RNA capture on the RNA capture material occurs in the presence of alcohol.

The separation module comprising the nucleic acid separation material is transferred to a second housing to recover DNA retained by the nucleic acid separation material. In one aspect, the nucleic acid separation material is washed with a solution comprising a chaotropic agent (e.g., for example, about 4M or greater in concentration). After DNA is eluted off of the separation module (e.g., with detergent), it can be further purified. For example, in one aspect, the DNA is purified using a polymeric membrane under low ionic strength and alcohol conditions, as described in U.S. patent application Ser. No. ______ (Attorney Docket No. 10050222-2), “Methods and Kits for DNA Purification on Polymeric Membranes at Low Ionic Strength” by Gerald Hall, et al. However, further purification may be done using any method known in the art, and does not necessarily require a solid phase (e.g., the DNA may be further purified by alcohol precipitation, for example).

In one embodiment, the separation module is contacted with an elution or releasing solution for eluting/releasing DNA from the nucleic acid separation material of the separation module into the collection module of the second housing. In one aspect, the elution/releasing solution comprises a low ionic strength solution comprising a surfactant, and may be an ionic detergent, such as 0.01-5% Sarcosyl (e.g., N-lauroylsarcosine). DNA eluted/released from the nucleic acid separation material can be collected in the second collection module by centrifugation or by the application of pressure or vacuum to the device or by gravity. In one aspect, 0.5 to 3 volumes of a low molecular weight alcohol, e.g., such as isopropanol is added to the DNA-containing flow through.

Precipitated RNA and precipitated DNA can be treated in parallel in these and subsequent steps. For example, generally, precipitated nucleic acids (i.e., RNA or genomic DNA) can be pelleted by centrifugation (e.g., a spin step of 10-30 minutes at room temperature at 16,000 g). Pelleted nucleic acids are resuspended, preferably after washing one or more times with a wash solution, for example, such as 60-90% ethanol. A buffer (i.e., 1-50 mM Tris ph 7-9) may also be included in the wash solution. After washing, pelleted nucleic acids are resuspended in a suitable buffer, for example, H₂O or TE. Additional, optional, purification steps may be added by contacting a DNA- or RNA-containing solution with additional DNA capture or RNA capture modules as needed. Further purification of RNA and/or DNA can be done by any method known in the art. e.g., using a solid phase for nucleic acid capture, such as a polymeric membrane, as described above, a silica-based solid phase, or without the use of a solid phase (e.g., such as by alcohol precipitation). RNA and DNA purification may be done by the same or by different methods. In certain aspects RNA is purified according to any of the methods described in U.S. Patent Publication 20050042660A1.

The quality and/or quantity of nucleic acids collected may be evaluated and optimized using methods well known in the art, such as obtaining an A260/A280 ratio, evaluating an electrophoresed sample, or by using Agilent Technologies® RNA 6000 Nano assay (part no. 50654476) on the Agilent Technologies® Bioanalyzer 2100 (part no. G2938B, Agilent Technologies®, Palo Alto, Calif.) as per manufacturer's instructions.

Simultaneous collection of both DNA and RNA from the same sample permits high throughput, parallel sample processing. For example, RNA may be collected from a sample for gene expression analysis while DNA may be collected from the same sample for genome analysis, such as comparative genomic hybridization or location analysis. In this way changes in the copy number of DNA in a sample, and/or binding patterns of proteins to DNA in a sample can be correlated with gene expression in that sample.

In one embodiment, the invention further provides kits. In one aspect, a kit according to the invention provides a device comprising a separation module comprising a nucleic acid separation material and one or more collection modules for collecting RNA, e.g., the collection modules are substantially RNase-free. In certain aspects, the nucleic acid separation material comprises a silica-based material or a polymeric material. In one aspect, the one or more collection modules further comprise an RNA capture material for retaining precipitated RNA molecules. In certain aspects, the RNA capture material comprises a polymeric material, such as a polysulfone-containing material. In one aspect, the RNA capture material does not comprise a silica-based material or an anion exchange material.

In another aspect, the kit comprises one or more of solutions for facilitating separation of DNA from RNA in a sample. In one aspect, the kit includes a lysis/nucleic acid (NA) separation buffer for facilitating nucleic acid separation on a NA separation material. In certain aspects, the lysis/NA separation solution comprises: a chaotropic salt, such as any of those described above. In one aspect, the lysis/NA separation solution does not comprise alcohol and/or comprises a pH of less than 8.0 (for example, the lysis/NA separation solution comprises a pH of from about pH 6.5 to about pH 7.5). In another aspect, the kit further comprises a wash buffer for washing the separation module, a DNA elution/releasing buffer (e.g., comprising a low ionic strength solution) for eluting DNA from the separation module. In certain aspects, the DNA elution/releasing buffer comprises a surfactant, such as an ionic detergent (e.g., N-lauroylsarcosine). In another aspect, the kit further comprises an RNA purification module to purify the RNA from the separation module flow through and a DNA purification module to purify DNA from the separation module eluate. In other aspect, the RNA purification and DNA purification releasing buffers comprises a low ionic strength solutions. However, in one aspect, the RNA purification and DNA purification releasing buffers do not comprise a surfactant, e.g., the RNA purification and DNA purification releasing buffers can comprise a nuclease-free buffer or even water.

In a further aspect, the kit comprises labeling reagents for labeling nucleic acid, primers and suitable polymerases for incorporating labels into a nucleic acid molecule, and the like. Complementary RNA (“cRNA”) also known as aRNA (amplified RNA), molecules can be synthesized and used as hybridization probes used to detect targets (usually DNA) in, for example, a microarray system. During the cRNA synthesis, label is incorporated into the cRNA molecule, for example, a fluorescent label such as cyanine or can be attached later to the cRNA molecule by many different enzymatic methods. RNA also may be converted to cDNA using methods known in the art. RNA, cRNA, or cDNA can be labeled, e.g., during or after an amplification step. Similarly, DNA can be labeled during or after an amplification step.

In one aspect, the kit comprises reagents for performing a CGH assay, e.g., such as reagents for performing a whole genome amplification reaction. Such reagents can include, but are not limited to: random primers, degenerate primers, primers that bind to universal adaptors or linker molecules, polymerases (e.g., such as phi29, the Klenow fragment of DNA pol I, etc), helicases, single-stranded binding proteins and the like. In still another aspect, the kit can comprise one or more arrays. Instructions for a practitioner to practice the invention may also be included. Array CGH assays may be performed as described in WO2004058945, for example. Such array assays can be performed in parallel or sequentially with gene expression assays on the same or different arrays. In still another aspect, one or more reagents for performing location analysis may be included in the kit, such as described in U.S. Pat. No. 6,410,243, for example.

### EXAMPLES

### Isolation of Total RNA and Genomic DNA From the Same Biological Sample

The following examples are provided to illustrate methods according to certain aspects of the invention and are not intended to limit the scope of the invention.

The protocol generally used was as follows:

Tissue is collected and processed immediately. Sample is weighed and placed in a tube containing 20 μl of Lysis Solution (4M guanidine isothiocyanate, 25 mM Tris pH7.5, 10 mM EDTA, 1% β-mercaptoethanol) per milligram of tissue or 10 ul of Plant Extraction Solution (5.5M guanidine hydrocloride, 50 mM Bis Tris pH6.6, 10 mM EDTA, 1% β-mercaptoethanol). The sample is immediately and vigorously homogenized using a conventional rotor-stator homogenizer with a stainless steel probe at 15,000 rpm. Up to 600 μl of homogenate (equivalent to 30 mg of tissue) is centrifuged through a mini-prefiltration column available in Agilent's Total RNA Isolation Mini Kit (Product No. 5185-6000) from Agilent Technologies, Inc. (Palo Alto, Calif.) for 3 minutes at full speed (for a typical microcentrifuge, approximately 16,000×g). The flow through is saved for RNA isolation and the prefiltration column is transferred to a clean 2 ml collection tube for isolation of genomic DNA.

RNA Isolation:

70% ethanol (100% isopropanol for plants) is added to the flow-through, using the volume equal to the amount of homogenate initially added to the prefiltration column. The solution is mixed until it appears homogeneous. The mixture may be incubated for 5 minutes at room temperature. The ethanol (isopropanol)/flow-through mixture (up to 700 μl) is contacted with a matrix for reversibly binding RNA such as an MMM column, available as the mini-isolation column in Agilent's Total RNA Isolation Mini Kit from Agilent Technologies, Inc. (Palo Alto, Calif.). Flow through from this column is discarded after centrifugation for 30 seconds at full speed. The RNA-loaded column is replaced in the collection tube and 500 μl of wash solution (25 mM Tris pH7.5, 80% ethanol) is added to the mini-isolation column which is centrifuged for 30 seconds at full speed. Flow-through is again discarded and the wash step is repeated for a total of two washes. The mini-isolation column is then spun for 2 minutes at full speed to completely remove trace amounts of wash solution. The mini-isolation column is transferred into a new 1.5 mol RNase-free final collection tube. 10-50 μl of nuclease free water is added to the top center of membrane (without touching the membrane). After 1 minute, the column in the final collection tube is centrifuged for 1 minute at full speed to collect RNA from the isolation column.

DNA Isolation:

400 μl of prefilter wash solution (0.5 M guanidine isothiocyanate, 80% ethanol) is added to the prefiltration column comprising genomic DNA and the column is centrifuged for 1 minute at maximum speed. The flow-through is discarded and DNA elution solution (1% sarcosyl) is added and the column is spun at maximum speed for three minutes. An equal volume of isopropanol is added to the eluate and the solution is mixed until it appears homogeneous. The mixture may be incubated for 5 minutes at room temperature. The isopropanol/eluate mixture (up to 700 μl) is added to a mini isolation column and centrifuged for 30 seconds at full speed. The flow-through is discarded and the DNA-loaded column is replaced in the collection tube. 500 μl of wash solution (25 mM Tris pH 7.5, 80% ethanol) is added to a mini isolation column (the same type which is used for RNA isolation) and the column is centrifuged for 30 seconds at full speed. The flow through is discarded, and the mini-isolation column is replaced in the same collection tube. The wash step is repeated for a total of two times and the column is spun for 2 minutes at full speed to completely remove trace amounts of wash solution. The mini isolation column is transferred into a new 1.5 ml final collection tube. 10-50 μl of nuclease-free water or TE is added to the top center of the membrane (without touching the membrane). After 1 minute, the column is centrifuged for 1 minute at full speed.

RNA isolation steps and DNA isolation steps can be processed in parallel since they are quite similar.

### Example 1

### Recovery of Tobacco Leaf Genomic DNA from 4-Layer Glass Fiber Prefilters with Various Elution Solutions

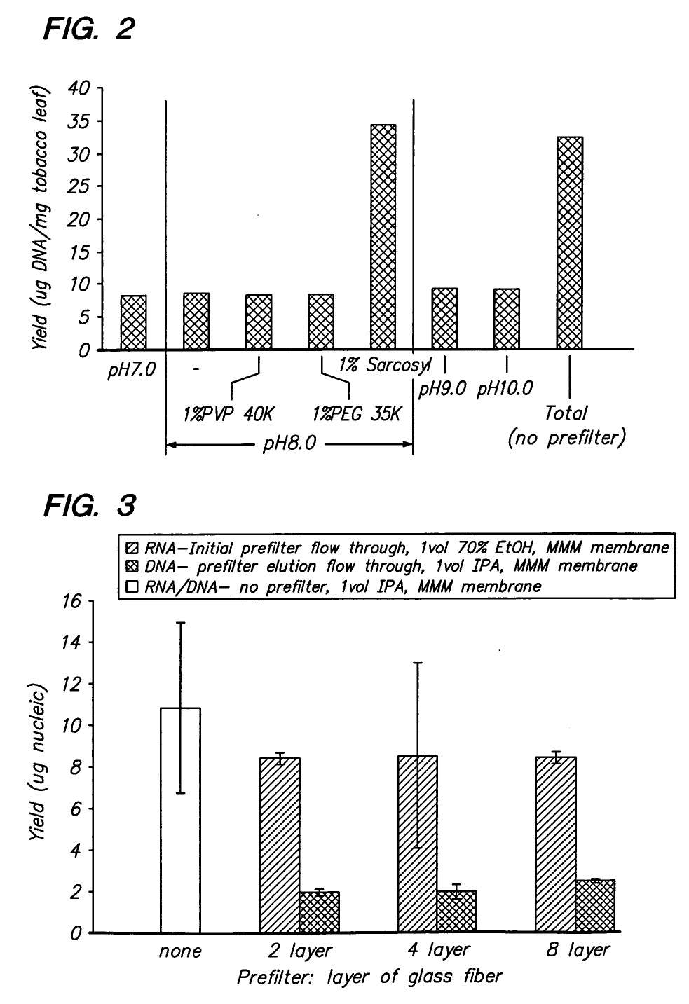

Tobacco leaf extracts were processed as described above (except for the Extraction Solution used was Plant Extraction solution and isopropanol was used to precipitate RNA). After the extract was passed through a 4-layer glass fiber prefilteration column, DNA was eluted with the various elution solutions shown in FIG. 2. A total nucleic acid sample was prepared by alcohol precipitation (no passage through a prefilter). Nucleic acid was quantified by spectrophotometry at 260 nm. An elution solution of 1% sarcosyl at pH 8 showed superior recovery from the prefiltration column.

### Example 2

### Recovery of RNA and DNA from Mouse Liver Extracts with Various Layers of Glass Fiber Prefilters

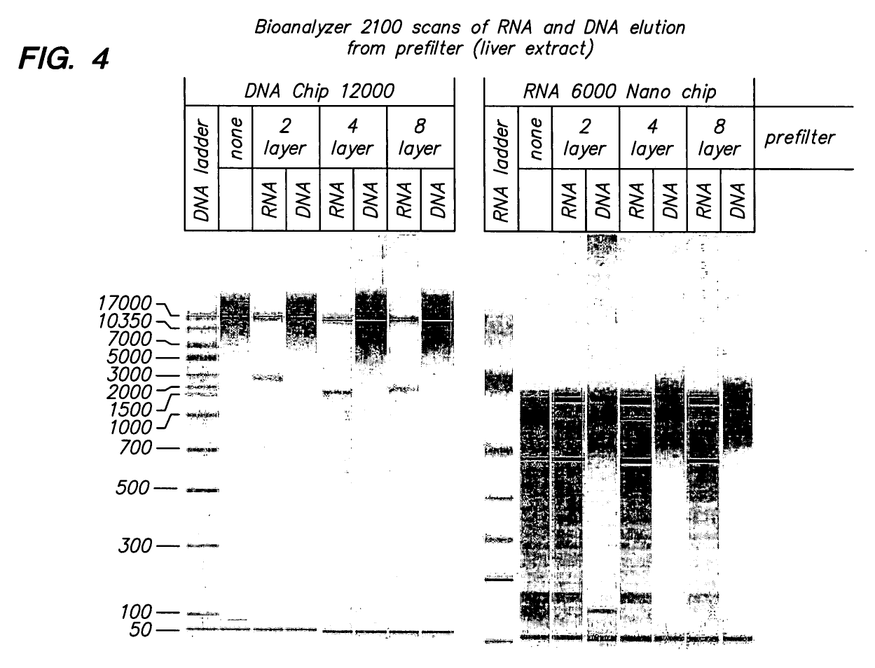

Mouse liver extracts were processed as described. Extracts were passed through a 2-, 4-, and 8-layer glass fiber prefiltration column. RNA and DNA were purified as described. A total nucleic acid sample was prepared by alcohol precipitation (i.e., no passage through a prefiltration column). Total nucleic acid yield (A260 nm) in the RNA and DNA fractions was determined and is shown in FIG. 3. FIG. 4 illustrates results of scans using the Agilent Bioanalyzer 2100 DNA Chip 12000 (left panel) and the RNA 6000 Nano Chip (right panel). The results show good recovery of genomic DNA and RNA in the appropriate fractions without significant contamination of genomic DNA in the RNA fraction or of RNA in the genomic DNA fraction.

### Example 3

### Recovery of Genomic DNA from Various Mouse Tissues Using 2-, 4- and 8-Layer Glass Fiber Prefilters

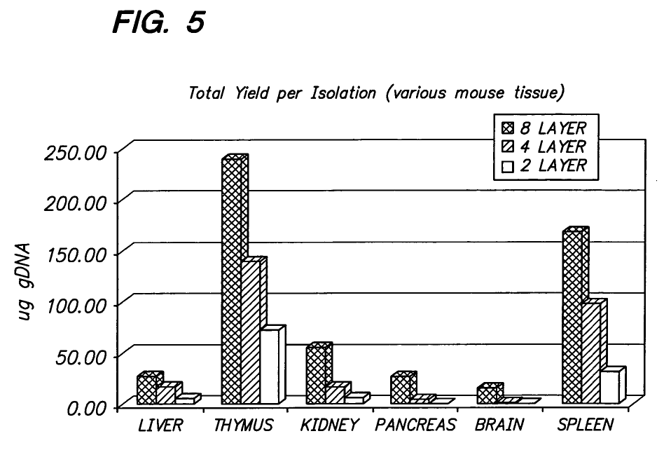

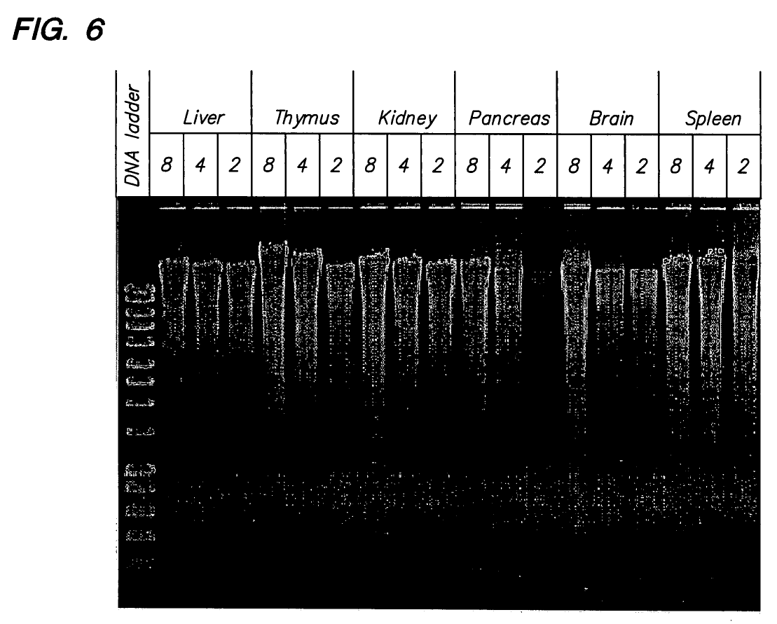

Mouse tissue extracts from liver, thymus, kidney, pancreas, brain and spleen were processed as described above. Extracts were passed through 2-, 4-, and 8-layer glass fiber prefiltration columns. DNA was purified as described above. As shown in FIG. 5, good yields of genomic DNA were obtained using each of the prefiltration columns, with higher yields obtained from the 8- and 4-layer columns compared to the 2-layer columns. As shown in FIG. 6, good yields of high molecular weight genomic DNA was obtained using each of the columns.

While this invention has been particularly shown and described with references to specific embodiments, it will be understood by those skilled in the art that various changes in form and details may be made therein without departing from the spirit and scope of the invention as defined by the appended claims.

References, patents, and patent applications cited herein are incorporated by reference in their entireties herein.

What is claimed is:

1. A method for separating RNA and DNA from a sample comprising
contacting a nucleic acid separation material with a sample comprising RNA and DNA, under conditions where DNA is captured by the nucleic acid separation material and RNA is not, wherein the separation conditions include a pH of less than 8.0;
removing the nucleic acid separation material from the sample;
releasing DNA from the nucleic acid separation material and purifying released DNA.

2. The method of claim 1, further comprising purifying RNA remaining in the sample.

3. The method of claim 1, wherein the DNA comprises genomic DNA.

4. The method of claim 1, wherein the separation conditions include contacting the sample to the nucleic acid separation material in the absence of alcohol.

5. The method of claim 1, wherein the nucleic acid separation material comprises one or more of: a silica-based solid phase material.

6. The method of claim 1, wherein DNA is released from the nucleic acid separation material by contacting the nucleic acid separation material with a detergent.

7. The method of claim 6, wherein the detergent is N-lauroylsarcosine.

8. The method of claim 7, wherein the concentration of N-lauroylsarcosine is about 0.01% to about 5% v/v.

9. The method of claim 1, wherein the released DNA is subjected to one or more purification procedures.

10. The method of claim 2, wherein the RNA remaining in the sample is subjected to one or more purification procedures.

11. The method of claim 9, wherein the purification is by precipitation or by contact with a nucleic acid capture material.

12. The method of claim 10, wherein the purification is by precipitation or by contact with a nucleic acid capture material.

13. The method of claim 11, wherein the nucleic acid capture material is a silica-based material.

14. The method of claim 12, wherein the nucleic acid capture material is a silica-based material.

15. The method of claim 11, wherein the nucleic acid capture material is a non-silica-based capture material.

16. The method of claim 12, wherein the nucleic acid capture material is a non-silica-based capture material.

17. The method of claim 15, wherein the nucleic acid capture material is a polymeric material.

18. The method of claim 16, wherein the nucleic acid capture material is a polymeric material.

19. The method of claim 17, wherein the polymeric material comprises polysulfone.

20. The method of claim 18, wherein the polymeric material comprises polysulfone.

21. The method of claim 19, wherein the polymeric material further comprises polyvinylpyrrolidone.

22. The method of claim 21, wherein the polymeric material further comprises polyvinylpyrrolidone.

23. The method of claim 11, further comprising releasing the nucleic acid from the nucleic acid capture material.

24. The method of claim 12, further comprising releasing the nucleic acid from the nucleic acid capture material.

25. The method of claim 1, wherein the sample comprises a biological sample and is homogenized prior to contacting with the nucleic acid separation material.

26. The method of claim 25, wherein the sample is homogenized in a solution comprising a chaotropic salt.

27. The method of claim 26, wherein the solution comprises at least about 4 M of a chaotropic salt.

28. The method of claim 27, wherein the chaotropic salt comprises guanidine isothiocyanate, guanidine HCl, sodium perchlorate, ammonium thiocyanate, sodium iodide, or a combination thereof.

29. The method of claim 1, wherein nucleic acid separation material is contacted with an ionic detergent for releasing the DNA.

30. A kit comprising a silica-based nucleic acid separation material and an ionic detergent.

31. The kit of claim 30, wherein the ionic detergent comprises N-lauroylsarcosine.

32. The kit of claim 30, further comprising a DNA capture material for purifying DNA.

33. The kit of claim 30, further comprising an RNA capture material for purifying RNA.

34. The kit of claim 30, further comprising a precipitating reagent for precipitating a nucleic acid.

35. The kit of claim 34, wherein the precipitating reagent comprises an alcohol.

36. The kit of claim 34, further comprising a DNA and/or RNA capture material.

37. The kit of claim 32, wherein the DNA capture material comprises a polymeric material.

38. The kit of claim 32, wherein the DNA capture material comprises a silica-based material.

39. The kit of claim 38, further comprising an RNA capture material.

40. The kit of claim 39, wherein the RNA capture material comprises a polymeric material.

41. The kit of claim 37, wherein the polymeric material comprises polysulfone.

42. The kit of claim 40, wherein the polymeric material comprises polysulfone.

43. The kit of claim 37, wherein the polymeric material further comprises polyvinylpyrrolidone.

44. The kit of claim 40, wherein the polymeric material further comprises polyvinylpyrrolidone.

45. The kit of claim 30, wherein the kit further comprises a chaotropic salt.

46. The kit of claim 45, wherein the chaotropic salt comprises guanidine isothiocyanate, guanidine HCl, sodium perchlorate, ammonium thiocyanate, sodium iodide, or a combination thereof.
